(19) United States
(12) Patent Application Publication
(10) Pub. No.: US 2023/0130574 A1
(43) Pub. Date: Apr. 27, 2023

(54) APPARATUS FOR DIAGNOSING PRESBYOPIA

(71) Applicant: THE CATHOLIC UNIVERSITY OF KOREA INDUSTRY-ACADEMIC COOPERATION FOUNDATION, Seoul (KR)

(72) Inventors: Yong Chan KIM, Seoul (KR); Kee Il LEE, Gyeongsangbuk-do (KR)

(21) Appl. No.: 17/910,455

(22) PCT Filed: Jan. 11, 2021
(86) PCT No.: PCT/KR2021/000291

§ 371 Date: Sep. 9, 2022

(30) Foreign Application Priority Data
Mar. 9, 2020 (KR) 10-2020-0028856

(51) Int. Cl. A61B 3/09; A61B 3/032

(52) U.S. Cl. CPC A61B 3/09; A61B 3/032; A61B 2090/061

(57) ABSTRACT

A presbyopia diagnosis apparatus includes: a measurement bar that is elongated along an axis in one direction, has a front end arranged to face the eyes of a subject for presbyopia measurement, and a rear end connected to a support, wherein a scale for measuring a distance is marked on the measurement bar; and a sliding unit that is movably installed on the measurement bar and has an installation groove formed therein, the installation groove for mounting a card for measuring presbyopia.

### CROSS REFERENCE TO RELATED APPLICATIONS AND CLAIM OF PRIORITY

This application claims benefit under 35 U.S.C. 119, 120, 121, or 365(c), and is a National Stage entry from International Application No. PCT/KR2021/000291 filed on Jan. 11, 2021, which claims priority to the benefit of Korean Patent Application No. 10-2020-0028856 filed in the Korean Intellectual Property Office on Mar. 9, 2020, the entire contents of which are incorporated herein by reference.

### BACKGROUND

### 1. Technical Field

The present disclosure relates to a presbyopia diagnosis apparatus, and more particularly, to a presbyopia diagnosis apparatus having an improved structure to enable diagnosis of presbyopia and accurately measure a presbyopia distance to reduce worsening of presbyopia symptoms and enable precise correction of eyesight.

### 2. Background Art

Presbyopia is an eye disorder involving the worsening of the ability to focus due to reduced elasticity of the crystalline lens according to aging, and is often felt in the early to mid-40s. Its symptoms include blurred vision along with impaired near vision.

Presbyopia may involve the delay of the change of focus when looking at distant and near objects alternately, eye fatigue and headaches when reading a book, and the symptoms worsen when the lighting is dim or when looking at small text, and the farther away the object to be viewed is, the more comfortable your eyes will see.

Presbyopia is diagnosed by confirming a patient's subjective symptoms through a questionnaire and performing visual acuity measurement. Generally, a patient is shown a card for vision measurement, but the measurement is made by manually changing the distance between the card and the patient. Therefore, there is a problem in that the accuracy of the measurement is low and accurate presbyopia distances cannot be calculated.

Korean Patent No. 10-1564589 discloses a technology for automatically adjusting the font size of an electronic device for measuring presbyopia, but still has a problem in that a presbyopia distance of a patient cannot be accurately calculated.

### SUMMARY

The present disclosure has been made to solve the above problems, and an object of the present disclosure is to provide a presbyopia diagnosis apparatus that enables precise diagnosis of presbyopia and precise measurement of a presbyopia distance, and improves the convenience and efficiency of diagnosis.

A presbyopia diagnosis apparatus used for presbyopia diagnosis, includes: a measurement bar that is elongated along an axis in one direction, has a front end arranged to face the eyes of a subject for presbyopia measurement, and a rear end connected to a support, wherein a scale for measuring a distance is marked on the measurement bar; and a sliding unit that is movably installed on the measurement bar and has an installation groove formed therein, the installation groove for mounting a card for measuring presbyopia.

The sliding unit may include: a main frame in which the installation groove is formed; a pressing member which is movably installed on the main frame and is brought into contact with or released from contact with the card for measuring presbyopia, the card being located in the installation groove, to fix the card for measuring presbyopia, in the installation groove without using an additional fixing means; and a spring which is installed on the main frame and elastically presses the pressing member toward the card for measuring presbyopia.

The pressing member may include a tapered guide surface to guide movement of the card for measuring presbyopia, to a lower portion of the installation groove.

The support may include: a support frame on which a motor is installed; a lifting frame which is installed on the support frame to be lifted or lowered, to which the measurement bar is connected, and which is screwed to a transfer screw rotated by driving of the motor; and a guide bar guiding vertical lifting movement of the lifting frame.

A presbyopia measuring apparatus used for presbyopia diagnosis, includes: a rotatable bar elongated along an axis in one direction, having a front end arranged to face the eyes of a subject for presbyopia measurement and a rear end connected, in a dynamic manner, to a motor installed on a support, and having a screw portion on an outer circumferential surface thereof; a sliding unit which is movably installed on the rotatable bar, has an installation groove for mounting a card for presbyopia measurement, and a nut portion that is engaged with the screw portion of the rotatable bar; and a guide bar which is arranged in parallel to the rotatable bar and is connected to the support and passes through the sliding unit to guide linear motion of the sliding unit.

A scale may be marked on the guide bar.

The guide bar may include a reflective portion formed at a position facing a front end of the rotatable bar at a distance from the front end, the reflective portion being formed of a light-reflective material, and the sliding unit may include a sensing module for irradiating light to the reflective portion and receiving the light to measure a relative distance to the reflective portion.

According to the presbyopia diagnosis apparatus according to an embodiment of the present disclosure configured as above, the following effects are expected: presbyopia may be diagnosed by moving a sliding unit having a card for measuring presbyopia, mounted therein, along the measurement bar, and thus presbyopia may be precisely diagnosed; and a presbyopia distance may be accurately measured by reading a scale of the measuring bar corresponding to a position of the sliding unit where the presbyopia diagnosis is conducted, and thus it may be possible to guide a patient to make himself or herself an effort to put an object in a well-visible position through presbyopia distance recognition to thereby suppress the worsening of presbyopia symptoms and furthermore enable precise vision correction.

### BRIEF DESCRIPTION OF THE DRAWINGS

FIG. 1 is a diagram illustrating a structure and a state of usage of a presbyopia diagnosis apparatus according to an embodiment of the present disclosure.

FIG. 2 is a perspective view illustrating some elements of an embodiment of the present disclosure.

FIG. 3 is an enlarged view of region III of FIG. 1.

FIG. 4 is a diagram illustrating a structure and a state of usage of a presbyopia measuring apparatus according to another embodiment of the present disclosure.

FIG. 5 is a diagram illustrating a structure and a state of usage of a presbyopia diagnosis apparatus according to another embodiment of the present disclosure.

FIG. 6 is a block diagram for describing the operating principle of a sensing module included in another embodiment of the present disclosure.

### DETAILED DESCRIPTION

In the following description, in order to clarify the understanding of the present disclosure, descriptions of well-known techniques for the features of the present disclosure will be omitted. Embodiments herein are detailed descriptions to help the understanding of the present disclosure, and are not intended to be limiting of the scope of the present disclosure. Accordingly, equivalent inventions performing the same functions as the present disclosure will also fall within the scope of the present disclosure.

In addition, in the following description, like symbols denote like elements, and unnecessary redundant descriptions and descriptions of well-known technologies will be omitted. In addition, the description of each embodiment of the present disclosure that overlaps with the description of the technology that is the background of the present disclosure will also be omitted.

Hereinafter, a presbyopia diagnosis apparatus according to an embodiment of the present disclosure is described with reference to the attached drawings.

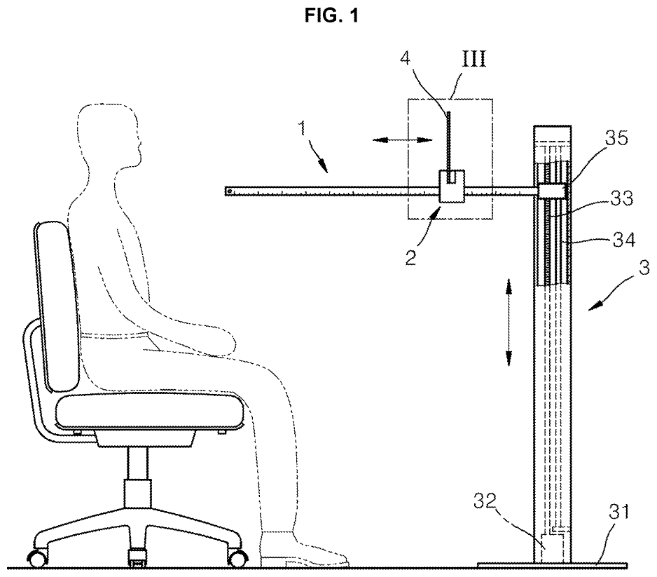

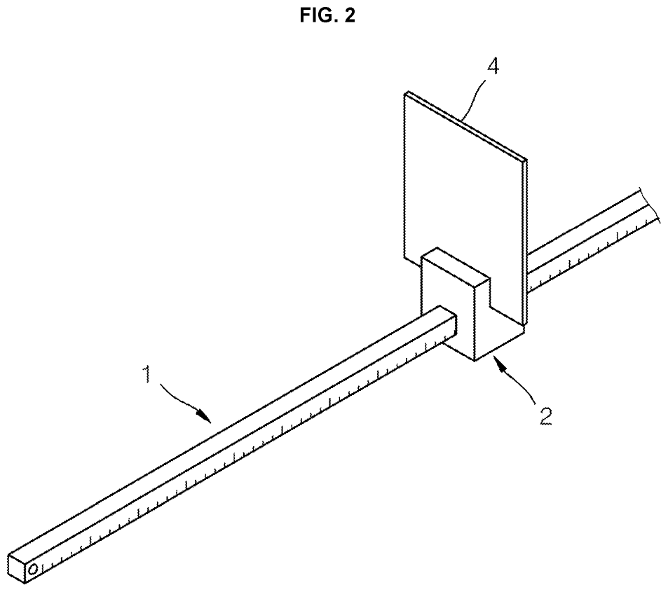

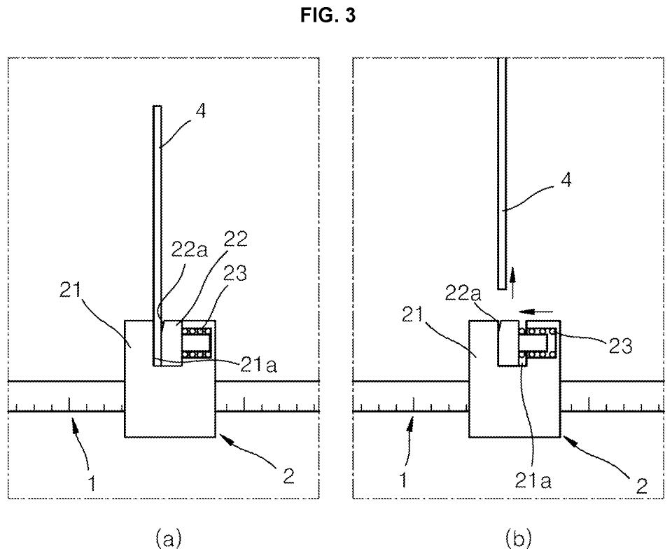

FIG. 1 is a diagram illustrating a structure and a state of usage of a presbyopia diagnosis apparatus according to an embodiment of the present disclosure. FIG. 2 is a perspective view illustrating some elements of an embodiment of the present disclosure. FIG. 3 is an enlarged view of region III of FIG. 1.

As well illustrated in FIGS. 1 and 2, a presbyopia diagnosis apparatus according to an embodiment of the present disclosure is used for diagnosing presbyopia, and includes a measurement bar 1 and a sliding unit 2.

The measurement bar 1 is elongated along an axis in one direction, and has a front end arranged to face the eyes of a subject for presbyopia measurement and a rear end connected to a support 3, and a scale for measuring a distance is marked thereon.

The sliding unit 2 is movably installed on the measurement bar 1 and has an installation groove 21a for mounting a card 4 for presbyopia measurement, and is relatively moved while having the card 4 mounted in the installation groove 21a, thereby enabling measurement of a relative distance of the measurement bar 1 with respect to a patient. Here, the card 4 for measuring presbyopia has letters and numbers of different sizes arranged vertically and horizontally, and generally has a thin thickness that is easy to hold and carry.

A distance between an actual patient and the card 4 for measuring presbyopia is measured obviously by summing a distance indicated on the scale of the measurement bar 1 corresponding to a position of the sliding unit 2 and a distance between the patient and the front end of the measurement bar 1. Here, a gap between the patient and the measurement bar 1 is narrow enough to be visually recognized, and thus, the distance between the patient and the measurement bar 1 may be easily measured.

According to the presbyopia diagnosis apparatus according to an embodiment of the present disclosure having a configuration as above, presbyopia may be diagnosed by moving the sliding unit 2 having the card 4 for measuring presbyopia, mounted therein, along the measurement bar 1, and thus, presbyopia may be precisely diagnosed; and a presbyopia distance may be accurately measured by reading a scale of the measuring bar 1 corresponding to a position of the sliding unit 2 where the presbyopia diagnosis is conducted, and thus it may be possible to guide a patient to make himself or herself an effort to put an object in a well-visible position through presbyopia distance recognition to thereby suppress the worsening of presbyopia symptoms and furthermore enable precise vision correction.

As well illustrated in FIG. 3, the sliding unit 2 included in the present embodiment includes a main frame 21, a pressing member 22, and a spring 23 to easily couple or remove the card 4 for measuring presbyopia.

That is, the main frame 21 is a portion in which the installation groove 21a is formed, and the pressing member 22 is movably installed on the main frame 21, when the pressing member 22 is moved in a direction to narrow a gap in the installation groove 21a, as illustrated in (a) of FIG. 3, the pressing member 22 is brought into contact with the card 4 for measuring presbyopia, located in the installation groove 21a, and when the pressing member 22 is moved in a direction to widen the gap in the installation groove 21a as illustrated in (b) of FIG. 3, the contact state is released.

The spring 23 is installed on the main frame 21 and elastically presses the pressing member 22 toward the card 4 for measuring presbyopia.

In the present embodiment having a configuration as above, the card 4 for measuring presbyopia is configured to be firmly fixed just by inserting the card 4 for measuring presbyopia, into the installation groove 21a of the sliding unit 2, by closely adhering the pressing member 22 to the card 4 for measuring presbyopia, by an elastic force by the spring 23. Thus, the card 4 for measuring presbyopia may be installed or removed without an additional fixing means or removal means, thereby further enhancing user convenience.

The pressing member 22 includes a tapered guide surface 22a to guide the movement of the card 4 for measuring presbyopia, to a lower portion of the installation groove 21a, such that the card 4 for measuring presbyopia is smoothly inserted into the installation groove 21a without interfering with the pressing member 22.

The support 3 included in the present embodiment includes a support frame 31, a lifting frame 35, and a guide bar 34 to adjust a height of the measurement bar 1 with respect to the ground.

The support frame 31 is a portion on which a motor 32 is installed, and the lifting frame 35 is a portion to which the measurement bar 1 is connected, and is installed on the support frame 31 to be lifted or lowered, and is screwed to a transfer screw 33 which is rotated by driving the motor 32. The guide bar 34 has a function of guiding vertical and linear motion of the lifting frame 35.

In the present embodiment having a configuration as described above, by operating the motor 32 to vertically lift or lower the lifting frame 35, to which the measurement bar 1 is connected, the height of the measurement bar 1 may be adjusted to match the eye position of a patient.

Hereinafter, a presbyopia measuring apparatus according to another embodiment of the present disclosure is described with reference to the attached drawings.

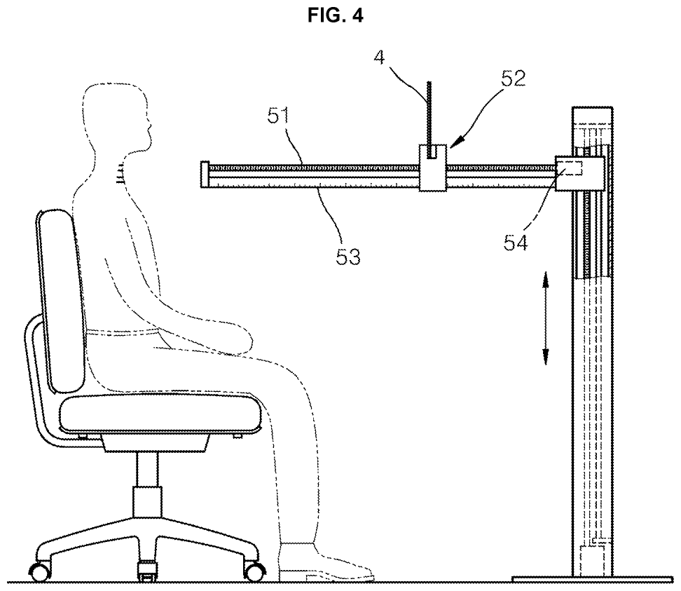

FIG. 4 is a diagram illustrating a structure and a state of usage of a presbyopia measuring apparatus according to another embodiment of the present disclosure.

According to the presbyopia measuring apparatus included in the present embodiment, a presbyopia distance of a patient may be precisely measured by automatically moving a sliding unit 52 in which a presbyopia measuring card is mounted, instead of moving manually. The presbyopia measuring apparatus includes a rotatable bar 51, the sliding unit 52, and a guide bar 53.

Like the measurement bar according to the embodiment described above, the rotatable bar 51 is elongated along an axis in one direction, and has a front end arranged to face the eyes of a subject for presbyopia measurement, but unlike the measurement bar according to the embodiment described above, a rear end of the rotatable bar 51 is connected, in a dynamic manner, to a motor 54 installed on a support, and a screw portion is provided on an outer circumferential surface of the rotatable bar 51.

The sliding unit 52 is movably installed on the rotatable bar 51, and an installation groove for mounting a card for presbyopia measurement is formed therein as in the above-described embodiment, but unlike the above-described embodiment, the sliding unit 52 includes a nut portion that is engaged with the screw portion of the rotatable bar 51.

In addition, according to the present embodiment, a guide bar 53 for guiding the sliding unit 52 such that the sliding unit 52 may perform a linear motion without rotating together with the rotatable bar 51 is included. That is, while being arranged in parallel to the rotatable bar 51, the guide bar 53 is connected to the support and passes through the sliding unit 52 to guide the linear motion of the sliding unit 52.

In the present embodiment having such a configuration, the sliding unit 52 in which a card for measuring presbyopia is mounted, is configured to be automatically moved by driving the motor 54, so that a change in a distance between the card for measuring presbyopia and a patient may be made minutely through precise control of the motor 54 to thereby enable precise measurement of a presbyopia distance, and also further enhance the user convenience.

As the guide bar 53 is marked with a scale, a user may easily measure a presbyopia distance by using the scale when the sliding unit 52 stops moving.

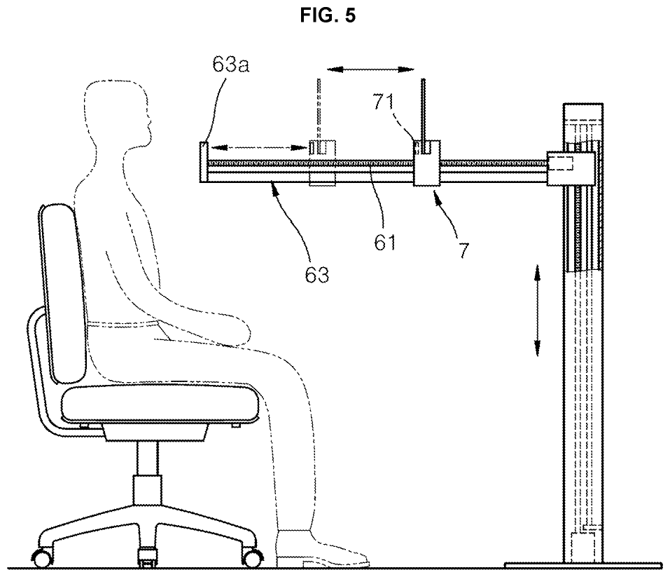

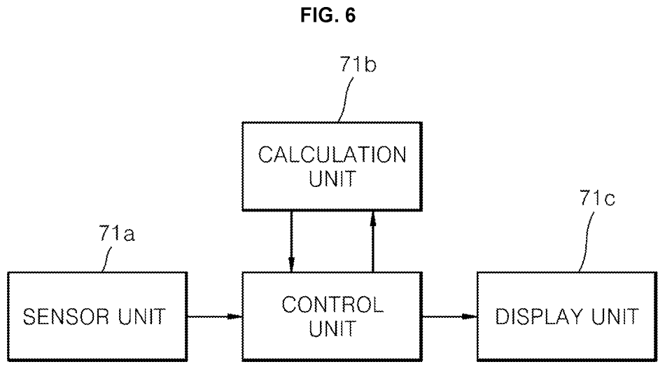

FIG. 5 is a diagram illustrating a structure and a state of usage of a presbyopia diagnosis apparatus according to another embodiment of the present disclosure. FIG. 6 is a block diagram for describing the operating principle of a sensing module included in another embodiment of the present disclosure.

As well illustrated in FIG. 5, a guide bar 63 included in the present embodiment includes a reflective portion 63a in addition to the elements of the guide bar (63) described above, and is not marked with a scale unlike the guide bar (63) described above.

That is, the reflective portion 63a is formed at a position facing a front end of a rotatable bar 61 at a distance from the front end, and is formed of a light-reflective material. A sliding unit 7 includes a sensing module for irradiating light to the reflective portion 63a and receiving the light to measure a relative distance to the reflective portion 63a.

According to the present embodiment having a configuration as above, as well illustrated in FIG. 5, the reflective portion 63a that allows reflection of light irradiated from the sensing module 71 installed in the sliding unit 7 is systemically coupled to the guide bar 63, and whether or not light is received and an arrival time of the light, or the like are sensed through a sensing unit 71a of the sensing module 71, and a calculating unit 71b measures a distance between the reflective portion 63a and the sliding unit 7 based on a sensed value, and a result of the measuring is displayed through a display unit 71c. Accordingly, the user does not have to read the scale to measure a presbyopia distance, and thus a presbyopia distance may be measured precisely and conveniently.

While various embodiments of the present disclosure have been described above, the present embodiment and the drawings attached to the present specification only clearly show a part of the technical idea included in the present disclosure, and it will be obvious that all modifications and specific embodiments that can be easily inferred by those skilled in the art within the scope of the technical spirit of the specification and the drawings of the present disclosure are included in the scope of the present disclosure.

1. A presbyopia diagnosis apparatus comprising:
a measurement bar that is elongated along an axis in one direction, measurement bar comprising a front end arranged to face eyes of a subject for presbyopia measurement and a rear end connected to a support, wherein a scale for measuring a distance is marked on the measurement bar; and
a sliding unit that is movably installed on the measurement bar and has an installation groove formed therein, the installation groove for mounting a card for measuring presbyopia.

2. The presbyopia diagnosis apparatus of claim 1, wherein the sliding unit comprises:
a main frame in which the installation groove is formed;
a pressing member which is movably installed on the main frame and is brought into contact with or released from contact with the card for measuring presbyopia, the card being located in the installation groove, to fix the card for measuring presbyopia, in the installation groove without using an additional fixing means; and
a spring which is installed on the main frame and elastically presses the pressing member toward the card for measuring presbyopia.

3. The presbyopia diagnosis apparatus of claim 2, wherein the pressing member includes a tapered guide surface to guide movement of the card for measuring presbyopia, to a lower portion of the installation groove.

4. The presbyopia diagnosis apparatus of claim 1, wherein the support comprises:
a support frame on which a motor is installed;
a lifting frame which is installed on the support frame to be lifted or lowered, to which the measurement bar is connected, and which is screw-coupled to a transfer screw rotated by driving of the motor; and
a guide bar which guides vertical lifting movement of the lifting frame.

5. A presbyopia measuring apparatus comprising:
a rotatable bar which is elongated along an axis in one direction, a rotatable bar comprising a front end arranged to face eyes of a subject for presbyopia measurement and a rear end connected, in a dynamic manner, to a motor installed on a support, and has a screw portion on an outer circumferential surface thereof;
a sliding unit which is movably installed on the rotatable bar, has an installation groove for mounting a card for presbyopia measurement, and a nut portion that is engaged with the screw portion of the rotatable bar; and
a guide bar which is arranged in parallel to the rotatable bar, and is connected to the support and passes through the sliding unit to guide linear motion of the sliding unit.

6. The presbyopia measuring apparatus of claim 5, wherein a scale is marked on the guide bar.

7. The presbyopia measuring apparatus of claim 5, wherein the guide bar comprises a reflective portion formed at a position facing a front end of the rotatable bar at a distance from the front end, the reflective portion being formed of a light-reflective material, and
the sliding unit includes a sensing module for irradiating light to the reflective portion and receiving the light to measure a relative distance to the reflective portion.
